(19) United States
(12) Patent Application Publication
(10) Pub. No.: US 2004/0197903 A1
(43) Pub. Date: Oct. 7, 2004

(54) Method for induction of proliferation of natural killer cells by dendritic cells cultured with GM-CSF and IL-15

(75) Inventor: Linda Pestano, Lynnwood, WA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Northwest Biotherapeutics, Inc., Bothell, WA (US)

(21) Appl. No.: 10/770,257

(22) Filed: Feb. 2, 2004

Related U.S. Application Data
(60) Provisional application No. 60/444,010, filed on Jan. 31, 2003.

(51) Int. Cl. C12N 5/08

(52) U.S. Cl. 435/372

(57) ABSTRACT

The present invention provides a dendritic cell that can induce the proliferation and activation of natural killer cells. The dendritic cells, designated NK dendritic cells, are characterized by the expression of increased levels of CD80, CD1a, and CD86 as compared to a mature dendritic cell cultured in the presence of granulocyte-macrophage colony stimulating factor (GM-CSF) and interleukin 4 (IL-4). Further, the dendritic cells are characterized by the expression of interleukin 12 (IL-12), tumor necrosis factor α (TNFα), and GM-CSF. The NK dendritic cells are produced by providing a cell population comprising low-adherence monocytic dendritic precursor cells that have been cultured in the presence of granulocyte-monocyte colony stimulating factor (GM-CSF) and interleukin 15 (IL-15) and by contacting the cells with an effective amount of a dendritic cell maturation agent. NK dendritic cells are capable of inducing at least a 10-fold increase in the number of NK cells typically found in a sample of peripheral blood.

### BACKGROUND OF THE INVENTION

Antigen presenting cells (APC) are important in eliciting an effective immune response. APC not only present antigens to T cells with antigen-specific receptors, but also provide the signals necessary for T cell activation. Such signals remain incompletely defined, but are known to involve a variety of cell surface molecules as well as cytokines or growth factors. The factors necessary for the activation of naive or unprimed T cells may be different from those required for the re-activation of previously primed memory T cells. Although monocytes and B cells have been shown to be competent APC, their antigen presenting capacities appear to be limited to the re-activation of previously sensitized T cells. Hence, they are not capable of directly activating functionally naive or unprimed T cell populations. On the other hand, dendritic cells are capable of both activating naive and previously primed T cells.

Dendritic cells are a heterogeneous cell population with a distinctive morphology and a widespread tissue distribution, including blood. (See, e.g., Steinman, Ann. Rev. Immunol. 9:271-96 (1991).) The cell surface of dendritic cells is unusual, with characteristic veil-like projections. Mature dendritic cells are generally identified as CD86⁺, CD3⁻, CD11c⁺, CD19⁻, CD83⁺ and HLA-DR⁺.

Dendritic cells process and present antigens, and stimulate responses from naive and unprimed T cells and memory T cells. In particular, dendritic cells have a high capacity for sensitizing MHC-restricted T cells and are very effective at presenting antigens to T cells, both self-antigens during T cell development and tolerance, and foreign antigens during an immune response. In addition to their role in antigen presentation, dendritic cells also directly communicate with non-lymph tissue and survey non-lymph tissue for an injury signal (e.g., ischemia, infection, or inflammation) or tumor growth. Once signaled, dendritic cells initiate an immune response by releasing cytokines that stimulate activity of lymphocytes and monocytes.

Due to their effectiveness at antigen presentation, there is growing interest in using dendritic cells as an immunostimulatory agent, both in vivo and ex vivo. Dendritic cell precursors have been isolated by various methods, such as, for example, density gradient separation, fluorescence activated cell sorting, immunological cell separation techniques such as panning, complement lysis, rosetting, magnetic cell separation techniques, nylon wool separation, and combinations of such methods. (See, e.g., O'Doherty et al., .J Exp. Med. 178:1067-76 (1993); Young and Steinman, J. Exp. Med. 171:1315-32 (1990); Freudenthal and Steinman, Proc. Natl. Acad. Sci. USA 87:7698-702 (1990); Macatonia et al., Immunol. 67:285-89 (1989); Markowicz and Engleman, J. Clin. Invest. 85:955-61 (1990).) Methods for immuno-selecting dendritic cells include, for example, using antibodies to cell surface markers associated with dendritic cell precursors, such as anti-CD34 and/or anti-CD14 antibodies coupled to a substrate. (See, e.g., Bernhard et al., Cancer Res. 55:1099-104 (1995); Caux et. al., Nature 360:258-61 (1992).)

The use of such isolated dendritic cells as immunostimulatory agents has been limited, however, due to the low frequency of dendritic cells in peripheral blood and the low purity of dendritic cells isolated by such methods. In particular, the frequency of dendritic cells in human peripheral blood has been estimated at about 0.1% of the white cells. Similarly, there is limited accessibility of dendritic cells from other tissues, such as lymphoid organs.

The low frequency of dendritic cells has increased interest in isolating cell population enriched in dendritic cell precursors, and culturing these precursors ex vivo to obtain enriched populations of immature or mature dendritic cells. Because the characteristics of dendritic cell precursors remain incompletely defined, current methods typically used for isolating dendritic cell precursors do not result in purified fractions of the desired precursors, but instead generally produce mixed populations of leukocytes enriched in dendritic cell precursors. In one example method, leukocytes are isolated by a leukapheresis procedure. Additional methods are typically used for further purification to enrich for cell fractions thought to contain dendritic cells and/or dendritic cell precursors. Similarly, methods such as differential centrifugation (e.g., isolation of a buffy coat) and filtration also produce a crude mixture of leukocytes containing dendritic cell precursors.

Another reported method for isolating dendritic cell precursors is to use a commercially treated plastic substrate (e.g., beads or magnetic beads) to selectively remove adherent monocytes and other “non-dendritic cell precursors.” (See, e.g., U.S. Pat. Nos. 5,994,126 and 5,851,756.) The adherent monocytes and non-dendritic cell precursors are discarded while the non-adherent cells are retained for ex vivo culture and maturation. In another method, apheresis cells were cultured in plastic culture bags to which plastic, i.e., polystyrene or styrene, microcarrier beads were added to increase the surface area of the bag. The cells were cultured for a sufficient period of time for cells to adhere to the beads and the non-adherent cells were washed from the bag. (Maffei, et al., Transfusion 40:1419-1420 (2000); WO 02/44338, incorporated herein by reference).

Subsequent to essentially all of the reported methods for the preparation of a cell population enriched for dendritic cells, the cell populations are typically cultured ex vivo for differentiation and/or expansion of the dendritic cells. Briefly, ex vivo differentiation typically has involved culturing the mixed cell populations enriched for dendritic cells in the presence of cellular growth factors, such as cytokines. For example, granulocyte/monocyte colony-stimulating factor (GM-CSF) and interleukin 4 (IL-4), Interleukin 7 (IL-7), or Interleukin 13 (IL-13), and the like, have been used to support and/or differentiate dendritic cells. The numbers of dendritic cells have also been expanded by culture in the presence of cytokines.

It has been previously disclosed that CD14⁺ monocytes cultured in the presence of IL-15 alone can be induced to mature into dendritic cells similar to monocytes cultured in GM-CSF plus IL-4 and TNFα. (Saikh et al., Clin. Exp. Immunol. 126:447-455 (2001); WO 02/40647). The effects on the monocytes was independent of endogenously produced GM-CSF and the IL-15 induced dendritic cells also expressed chemokines and stimulated strong allo-responses in T cells characteristic of mature dendritic cells. Monocytes have also been cultured ex vivo in the presence of GM-CSF and IL-15. (Mohamadzadeh et al., J. Exp. Med. 194:1013-1019 (2001); WO 01/85920).

The effectiveness of such ex vivo differentiation and expansion has been limited, however, by the quality of the starting population enriched in dendritic cells. Under some culture conditions, populations of dendritic cells and dendritic cell precursors that are heavily contaminated with neutrophils, macrophages and lymphocytes, or combinations thereof, can be overtaken by the latter cells, resulting in a poor yield of dendritic cells. Culture of dendritic cells containing large numbers of neutrophils, macrophages and lymphocytes, or combinations thereof, are less suitable for use as immunostimulatory preparations.

Once expanded, immature or mature dendritic cells have been exposed to a target antigen(s) to provide activated mature dendritic cells. In general, the antigen has been added to immature or mature dendritic cells under suitable culture conditions. In the case of immature dendritic cells, the cells are then allowed sufficient time to take up and process the antigen, and express antigenic peptides on the cell surface in association with either MHC class I or class II markers. Antigen can be presented to immature dendritic cells on the surface of cells, in purified form, in a semi-purified form, such as an isolated protein or fusion protein (e.g., a GM-CSF-antigen fusion protein), as a membrane lysate, as a liposome-protein complex, and other methods. In addition, as mature dendritic cells are inefficient at taking up and processing antigen, peptides that bind to MHC class I or MHC class II molecules can be added to mature dendritic cells for presentation.

Once activated dendritic cells are obtained, they can be administered to a patient to stimulate an immune response. Activated dendritic cells can be administered to a patient by bolus injection, by continuous infusion, sustained release from implants, or other suitable techniques known in the art. The activated dendritic cells also can be co-administered with physiologically acceptable carriers, excipients, buffers and/or diluents. Further, activated dendritic cells can be used to activate T cells, e.g., cytotoxic T cells, ex vivo using methods well known to the skilled artisan. The antigen specific cytotoxic T cells can then be administered to a patient to treat, for example, a growing tumor, a bacterial, or a viral infection.

Natural Killer (NK) cells typically comprise approximately 10 to 15% of the mononuclear cell fraction in normal peripheral blood. Historically, NK cells were first identified by their ability to lyse certain tumor cells without prior immunization or activation. NK cells are thought to provide a “back up” protective mechanism against viruses and tumors that might escape the CTL response by down regulating MHC class I presentation. In addition to being involved in direct cytotoxic killing, NK cells also serve a critical role in cytokine production, which may be important to control cancer, infection and may also be involved in fetal implantation. The ex vivo manipulation of NK cells, e.g., educating NK cells by culturing in the presence of an antigen, has been suggested for the treatment of inflammatory conditions.

Examination of the cell surface proteins expressed by NK cells has revealed that NK cells lack the T-cell receptor complex (TCR) and are CD3 negative. They also express several cell surface proteins specific to NK cells in the peripheral blood. These surface proteins include neural adhesion molecule (NCAM, CD56) among others.

Activated NK cells produce a variety of regulatory cytokines including TGFβ-1, IFNγ, TNFα, IL-1β, granulocyte-colony stimulating factor (G-CSF), and GM-CSF. Transfer of activated NK cells of the same donor type has been used in allogenic bone marrow transplantation to improve marrow engraftment without inducing graft versus host disease (GvHD). Administration of IL-2 activated NK cells has also been demonstrated to reduce GvHD in recipients of a bone marrow transplant supplemented with allogeneic T cells to inhibit the occurrence of GvHD without reducing the graft versus tumor cell effects in tumor bearing mice. (Asai et al., J. Clin. Invest. 101:1835-1842 (1998)).

Typically, NK cells can be selected using positive selection with an antibody specific for CD56 followed by negative selection with a monoclonal antibody specific for CD3 to deplete T cells. Following this procedure the cell population has been enriched in NK cells to about 98.6% of the cell population with a four-log depletion of T cells. (Naume et al., J. Immunol. Methods 136:1-9 (1991); Lang et al., Bone Marrow Transplant. 29:497-502 (2001). These cells can be cultured in the presence of IL-2 to ensure NK cell activation prior to administration to a patient. In other methods, a patient can be administered IL-2 to up regulate the proliferation of endogenous NK cells. Both immature and mature dendritic cells cultured in a mixture of GM-CSF and IL-4 have been demonstrated to activate resting human NK cells. Within one week of treatment with dendritic cells the NK cells were typically increased two- to four-fold in number, and started secreting IFNγ, as well as acquiring cytolytic activity against typical NK cell target cells. (Ferlazzo et al., J. Exp. Med. 195:343-351 (2002).

Unexpectedly the present invention provides compositions and methods for generating large numbers of activated NK cells from peripheral blood mononuclear cells. Monocytic dendritic precursor cells are isolated by minimal adherence to glass coated microcarrier beads to produce a population comprising low-adherence monocytic dendritic precursor cells. The low-adherence monocytic dendritic cell precursors are grown in serum-free media supplemented with GM-CSF and IL-15 to mature the low-adherence dendritic cells (NK dendritic cells). Mature low-adherence dendritic cells obtained by this process (NK dendritic cells) appear to be the same as typical mature dendritic cells based on visual inspection and analysis of cell surface markers and cytokine expression characteristic of dendritic cells isolated by other means. But, co-culture of the mature low-adherence dendritic cells (NK dendritic cells) with peripheral blood mononuclear cells (PBMC) results in the expansion of large numbers of natural killer (NK) cells instead of CD3⁺ T cells as typically found with co-culture of PBMC with monocytic dendritic cell precursors matured in the presence of GM-CSF and IL-4.

### BRIEF SUMMARY OF THE INVENTION

The present invention provides a dendritic cell that can induce the activation and proliferation of natural killer cells. Dendritic cells of the invention can be characterized by the expression of increased levels of CD80, and CD86 as compared to a mature dendritic cell cultured in the presence of granulocyte-macrophage colony stimulating factor (GM-CSF) and interleukin 4 (IL-4). Further, the dendritic cells are characterized by the expression of an increased level of CD1a on the surface of the cell and by the expression of interleukin 12 (IL-12), tumor necrosis factor α (TNFα), interleukin 1β (IL-1β), and GM-CSF.

In an embodiment of the present invention the NK dendritic cells of the present invention have been found to be capable of inducing at least a 10-fold increase in the number of NK cells present at the beginning of a co-culture of dendritic cells in a peripheral blood sample over an approximately 7 to 9 day period. In one specific embodiment of the present invention the number of NK cells were increased by up to about 30 to about 50 fold.

NK dendritic cells of the present invention are produced from a population of low-adherence dendritic cells by contacting the low-adherence monocytic dendritic precursor cells with an effective amount of granulocyte-monocyte colony stimulating factor (GM-CSF) and interleukin 15 (IL-15) to form low-adherence immature dendritic cells. The low-adherence immature dendritic cells are then contacted with an effective amount of a dendritic cell maturation agent under culture conditions suitable for maturation. Dendritic cell maturation agents suitable for use in the present invention include, but are not limited to, Bacillus Calmette-Guerin (BCG), lipopolysaccharide (LPS), TNFα, Interferon gamma (IFNγ), and combinations thereof. In a particular embodiment of the present invention a combination of BCG and IFNγ is used to mature the NK dendritic cells.

The low-adherence dendritic cells can be substantially purified from peripheral blood mononuclear cells (PBMCs). The cells are contacted with a substrate with a high affinity for dendritic precursor cells, such as glass coated microcarrier beads, in the presence of a high concentration of protein. A certain subpopulation of monocytic dendritic precursors cells adhere to the solid substrate with a low avidity while the remaining cells are separated from the solid surface. The low-adherence dendritic precursor cells are then isolated and cultured in the presence of GM-CSF and IL-15. The NK dendritic cells are characterized by the expression of increased levels of CD80, and CD86 as compared to a mature dendritic cell cultured in the presence of granulocyte-macrophage colony stimulating factor (GM-CSF) and interleukin 4 (IL-4). Further, the method produces NK dendritic cells that can be characterized by the expression of an increased level of CD1a on the surface of the cells and by the expression of interleukin 12 (IL-12), tumor necrosis factor α (TNFα), interleukin 1β (IL-1β), and GM-CSF. The NK dendritic cells can be subsequently cryopreserved. Still further, the dendritic cells can be contacted with NK cells in vivo, ex vivo, or in vitro. When the method is practices either ex vivo, or in vitro, the NK cells can be substantially isolated or provided as a population of, for example, leukocytes, such as peripheral blood monocytic cells (PBMCs).

In one particular embodiment of the present invention the NK dendritic cells are co-administered with antigen presenting dendritic cells to both induce the proliferation and activation of NK cells as well as induce an antigen specific T cell response.

### BRIEF DESCRIPTION OF THE DRAWINGS

FIGS. 1A and 1B depict the yield of dendritic cells after culture in GM-CSF alone, GM-CSF and IL-4, or GM-CSF and IL-15. FIG. 1A depicts the absolute number of monocytes at day 0 and the number of immature dendritic cells subsequent to 5 days of culture. FIG. 1B depicts the average yield as a percentage of total cell input.

FIGS. 2A and 2B depict a comparison of the yield of immature and mature dendritic cells after 5 days of culture in either GM-CSF alone, GM-CSF and IL-4, or GM-CSF and IL-15 followed by a one day maturation in BCG and IFNγ. FIG. 2A depicts the comparison of the number of monocytes at the beginning of the culture with the number of immature dendritic cells after 5 days of culture. FIG. 2B depicts a comparison of the number of immature dendritic cells at the beginning of maturation (2.5×10⁶ cells) with the number of mature dendritic cells following 20 h in the presence of BCG and IFNγ.

FIGS. 3A through 3C depict the ability of dendritic cells generated in various cytokines to stimulate T cell and/or NK cell expansion from peripheral blood mononuclear cells (PBMCs) during an 8 to 9 day co-culture. FIG. 3A depicts the percentage of CD3⁺ and CD16⁺ cells as an average from 4 separate co-cultures. FIG. 3B depicts the absolute number of CD3⁺ and CD 16⁺ cells in the co-cultures by day eight. FIG. 3C depicts the combined averages of seven similar experiments using four different donors.

FIG. 4 depicts the absolute number of CD3⁺ and CD16⁺ cells obtained when low-adherence dendritic cells generated in various cytokines are co-cultured with autologous PBMCs.

FIG. 5 depicts the number of CD3⁺ and CD16⁺ cells present in a mixed population of cells enriched for low-adherence dendritic cells subsequent to differentiation in in GM-CSF alone, GM-CSF and IL-4, or GM-CSF and IL-15 for 5 days. The differentiated dendritic cells from each culture condition were matured with BCG and IFNγ for 20 additional hours, followed by continued culture in T cell culture media in the presence of IL-2 and IL-15. The absolute number of each cell population was calculated by multiplying the percentage of CD3 or CD 16⁺ cell found in each cell line by the absolute number of cell in each coculture.

FIGS. 6A and 6B depict the ability of low adherence dendritic cell precursors cultured in the presence of GM-CSF, GM-CSF and IL-4, or GM-CSF and IL-15 to induce antigen specific T cells and NK cells subsequent to antigen loading and maturation. FIG. 6A depicts the absolute number of CD3⁺ cells and CD16⁺ cells induced by the dendritic cells from each culture condition. FIG. 6B depicts the number of Vβ 17/CD8⁺ T cells induced by the mature antigen loaded dendritic cells from each culture condition.

FIG. 7 depicts the percentage of cells expressing CD3 or CD56 that result when dendritic cells generated either in GM-CSF and IL-4 or GM-CSF and IL-15 are mixed, antigen loaded with influenza peptide M1-A4, and co-cultured with autologous PBMCs.

FIG. 8 depicts the ratio of dendritic cells generated with GM-CSF, GM-CSF and IL-4, or GM-CSF and IL-15 migrating towards the chemokine MIP-3β versus background migration (no MIP-3β).

FIGS. 9A and 9B depict the functional abilities of the NK cells induced by co-culture with dendritic cells generated with GM-CSF, GM-CSF and IL-4, or GM-CSF and IL-15. FIG. 9A depicts the percentage lysis of K562 tumor cells by each group of dendritic cells. FIG. 9B depicts the percentage of CD3⁺ cells and CD16⁺ cells in each dendritic cell population.

### DETAILED DESCRIPTION OF THE INVENTION

The present invention provides methods for inducing maturation of immature dendritic cells (DC) and for using those cells to induce the production of a substantial number of NK cells from a population of polymorphonuclear cells. Such cell populations include immature monocytic dendritic cells generated through culture of the DC precursors with GM-CSF and IL-15 followed by contact with a dendritic cell maturation factor, such as BCG and IFNγ, optionally with a predetermined antigen under suitable maturation conditions. The immature dendritic cells can be contacted with the antigen during or prior to maturation. Alternatively, immature monocytic dendritic cells, already exposed to antigen (e.g., in vivo), can be contacted with BCG and IFNγ under suitable maturation conditions. In a related aspect, compositions are provided as a maturation agent for immature dendritic cells that can induce the activation and proliferation of a substantial number of NK cells from peripheral blood mononuclear cells or NK cells found in the DC precursor preparation.

Monocytic dendritic cell precursors can be obtained from any tissue where they reside, particularly lymphoid tissues such as the spleen, bone marrow, lymph nodes and thymus. Monocytic dendritic cell precursors also can be isolated from the circulatory system. Peripheral blood is a readily accessible source of monocytic dendritic cell precursors. Umbilical cord blood is another source of monocytic dendritic cell precursors. Monocytic dendritic cell precursors can be isolated from a variety of organisms in which an immune response can be elicited. Such organisms include, for example, humans, and non-human animals, such as, primates, mammals (including dogs, cats, mice, and rats), birds (including chickens), as well as transgenic species thereof.

In certain embodiments, the monocytic dendritic cell precursors and/or immature dendritic cells can be isolated from a healthy subject or from a subject in need of immunostimulation, such as, for example, a prostate cancer patient or other subject for whom cellular immunostimulation can be beneficial or desired (i.e., a subject having a bacterial, viral or parasitic infection, and the like). Dendritic cell precursors and/or immature dendritic cells also can be obtained from an HLA-matched healthy individual for administration to an HLA-matched subject in need of immunostimulation.

Dendritic Cell Precursors and Immature Dendritic Cells

Methods for isolating dendritic cell precursors and immature dendritic cells from various sources, including blood and bone marrow, are known in the art. For the present invention dendritic cell precursors and immature dendritic cells are obtained by, for example, adherence to a monocyte binding substrate, such as, glass beads or to a glass coated plastic polystyrene microcarrier in the presence of a high concentration of protein, and the elution of those dendritic precursor cells that have weakly adhered to the surface of the substrate with PBS and EDTA.

Dendritic cell precursors and immature dendritic cells can be prepared in a closed, aseptic system. As used herein, the terms “closed, aseptic system” or “closed system” refer to a system in which exposure to non-sterile, ambient, or circulating air or other non-sterile conditions is minimized or eliminated. Closed systems for isolating dendritic cell precursors and immature dendritic cells generally exclude density gradient centrifugation in open top tubes, open air transfer of cells, culture of cells in tissue culture plates or unsealed flasks, and the like. In a typical embodiment, the closed system allows aseptic transfer of the dendritic cell precursors and immature dendritic cells from an initial collection vessel to a sealable tissue culture vessel without exposure to non-sterile air.

In certain embodiments, monocytic dendritic cell precursors are isolated by adherence to a monocyte-binding substrate, as disclosed in International Patent Application No. PCT/US02/23865 filed Jul. 25, 2002, the disclosure of which is incorporated by reference herein. For example, a population of leukocytes (e.g., isolated by leukopheresis) can be contacted with a monocytic dendritic cell precursor adhering substrate, e.g., a glass coated microcarrier bead, in the presence of a blocking agent that prevents non-specific binding as well as reduces the binding avidity of the monocytic dendritic cell precursor cells. When the population of leukocytes is contacted with the substrate, the monocytic dendritic cell precursors in the leukocyte population preferentially loosely adhere to the substrate. Other leukocytes (including other potential dendritic cell precursors) exhibit reduced binding affinity to the substrate, thereby allowing a subset of the monocytic dendritic cell precursors to be preferentially enriched on the surface of the substrate. Subsequent to cell binding and removal of non-adherent cells, the subset of monocytic dendritic cell precursors, referred to as “low-adherence” monocytic dendritic precursor cells, are eluted from the substrate by a buffered salt solution supplemented with a non-toxic chelating agent. By “non-toxic chelating agent” is intended those chelating agents that do not substantially reduce the viability of the monocytic dendritic cell precursors, for example, EDTA.

Suitable substrates include, for example, those having a large surface area to volume ratio, such as glass beads or a glass coated microcarrier. Such substrates can be, for example, a particulate or fibrous substrate. Suitable particulate substrates include, for example, glass particles, glass-coated plastic particles, glass-coated polystyrene particles, and other beads suitable for protein absorption. Suitable fibrous substrates include glass or glass coated microcapillary tubes and microvillous membrane. The particulate or fibrous substrate usually allows the adhered monocytic dendritic cell precursors to be eluted without substantially reducing the viability of the adhered cells. A particulate or fibrous substrate can be substantially non-porous to facilitate elution of monocytic dendritic cell precursors or dendritic cells from the substrate. A “substantially non-porous” substrate is a substrate in which at least a majority of pores present in the substrate are smaller than the cells to minimize entrapping cells in the substrate.

Adherence of the monocytic dendritic cell precursors to the substrate can optionally be enhanced by addition of binding media Suitable binding media include monocytic dendritic cell precursor culture media (e.g., AIM-V®, RPMI 1640, DMEM, X-VIVO-15®, and the like) supplemented, individually or in any combination, with for example, cytokines (e.g., Granulocyte/Macrophage Colony Stimulating Factor (GM-CSF), Interleukin 15 (IL-15), blood plasma, serum (e.g., human serum, such as autologous or allogeneic sera), purified proteins, such as serum albumin, divalent cations (e.g., calcium and/or magnesium ions) and other molecules that aid in the specific adherence of monocytic dendritic cell precursors to the substrate, or that prevent adherence of non-monocytic dendritic cell precursors to the substrate. In certain embodiments, the blood plasma or serum can be heated-inactivated. The heat-inactivated plasma can be autologous or heterologous to the leukocytes.

Isolated low-adherence monocytic dendritic cell precursors are cultured ex vivo for differentiation, maturation and/or expansion. (As used herein, isolated immature dendritic cells, dendritic cell precursors, T cells, and other cells, refers to cells that, by human hand, exists apart from their native environment, and are therefore not a product of nature. Isolated cells can exist in purified form, in semi-purified form, or in a non-native environment.) Briefly, ex vivo differentiation typically involves culturing the low-adherence dendritic cell precursors, or populations of cells comprising low-adherence dendritic cell precursors, in the presence of one or more differentiation agents. In particular, the differentiation agent in the present invention is a combination of granulocyte-macrophage colony stimulating factor (GM-CSF) and Interleukin 15 (IL-15). In certain embodiments, the low-adherence monocytic dendritic cell precursors are differentiated to form monocyte-derived immature low-adherence dendritic cells capable of inducing the activation and proliferation of a substantial number of natural killer cells after maturation. These immature low-adherence dendritic cells are referred to herein as “immature NK dendritic cells.”

The low-adherence dendritic cell precursors can be cultured and differentiated in suitable culture conditions. Suitable tissue culture media include AIM-V®, RPMI 1640, DMEM, X-VIVO-15®, and the like. The tissue culture media can be supplemented with serum, amino acids, vitamins, GM-CSF and IL-15, divalent cations, and the like, to promote differentiation of the cells into NK dendritic cells. In certain embodiments, the dendritic cell precursors can be cultured in serum-free media. Such culture conditions can optionally exclude any animal-derived products. Typically, the cytokines are added to the culture medium at a concentration of about 500 units/ml of GM-CSF and about 100 ng/ml of IL-15. Low-adherence dendritic cell precursors, when differentiated to form immature low-adherence dendritic cells, are phenotypically similar to skin Langerhans cells and demonstrate a typical expression pattern of cell surface proteins seen for immature monocytic dendritic cells, e.g., the cells are typically CD14^{-/low}, HLA-DR⁺, CD11c⁺, and express low levels of CD86 and CD80. In addition, the immature low-adherence dendritic cells are able to capture soluble antigens via specialized endocytosis.

The immature low-adherence dendritic cells can be matured to form mature low-adherence dendritic cells, also referred to as mature NK dendritic cells (NK DCs). Mature low-adherence DCs lose the ability to take up antigen and the cells display up-regulated expression of co-stimulatory cell surface molecules and secrete various cytokines. Specifically, mature low-adherence DCs express higher levels of MHC class I and II antigens and are generally identified as CD80⁺, CD86⁺, and CD14^{-/low}. Increased MHC expression leads to an increase in antigen density on the DC surface, while up regulation of co-stimulatory molecules CD80 and CD86 strengthens the T cell activation signal through the counterparts of the co-stimulatory molecules, such as CD28 on the T cells.

Mature low-adherence dendritic cells (NK DCs) can be prepared (i.e., matured) by contacting the immature low-adherence dendritic cell precursors that have been cultured in the presence of GM-CSF and IL-15 with an effective amount or concentration of a dendritic cell maturation agent. Dendritic cell maturation agents can include, for example, BCG, IFNγ, LPS, TNFα, and the like. Effective amounts of BCG typically range from about 10⁵ to 10⁷ cfu per milliliter of tissue culture media. Effective amounts of IFNγ typically range from about 100-1000 U per milliliter of tissue culture media. Bacillus Calmette-Guerin (BCG) is an avirulent strain of M. bovis. As used herein, BCG refers to whole BCG as well as cell wall constituents, BCG-derived lipoarabidomannans, and other BCG components that are associated with induction of a type 2 immune response. BCG is optionally inactivated, such as heat-inactivated BCG, formalin-treated BCG, and the like.

The immature low-adherence DCs are typically contacted with effective amounts of BCG and IFNγ for about one hour to about 24 hours. The immature dendritic cells can be cultured and matured in suitable maturation culture conditions. Suitable tissue culture media include AIM-V®, RPMI 1640, DMEM, X-VIVO 15®, and the like. The tissue culture media can be supplemented with amino acids, vitamins, cytokines, such as GM-CSF and/or IL-15, divalent cations, and the like, to promote maturation of the cells. A typical cytokine combination is about 500 units/ml of GM-CSF and 100 ng/ml of IL-15.

Maturation of NK dendritic cells can be monitored by methods known in the art for dendritic cells. Cell surface markers can be detected in assays familiar to the art, such as flow cytometry, immunohistochemistry, and the like. The cells can also be monitored for cytokine production (e.g., by ELISA, another immune assay, or by use of an oligonucleotide array).

Typically, immature NK dendritic cells can be cultured in the presence of BCG, IFNγ and a predetermined antigen under suitable maturation conditions to provide for dendritic cells that have processed the antigen and present the processed antigen on the surface of the mature dendritic cell. Optionally, the immature NK dendritic cells can be admixed with the predetermined antigen in a typical dendritic cell culture media without GM-CSF and IL-4, or a maturation agent. Following at least about 10 minutes to 2 days of culture with the antigen, the antigen can be removed and culture media supplemented with BCG and IFNγ can be added. These antigen presenting dendritic cells are typically admixed with PBMCs in vitro or ex vivo to stimulate the upregulation of a T helper or antigen specific cytotoxic T cell response. Cytokines (e.g., GM-CSF and IL-15) can also be added to the maturation media. Methods for contacting dendritic cells with antigen are generally known in the art. (See generally Steel and Nutman, J. Immunol. 160:351-60 (1998); Tao et al., J. Immunol. 158:4237-44 (1997); Dozmorov and Miller, Cell Immunol. 178:187-96 (1997); Inaba et al., J Exp Med. 166:182-94 (1987); Macatonia et al., J Exp Med. 169:1255-64 (1989); De Bruijn et al., Eur. J. Immunol. 22:3013-20 (1992); the disclosures of which are incorporated by reference herein.). In the present invention, the NK dendritic cells can also be admixed with BCG and IFNγ with or without a predetermined antigen to form a mature activated dendritic cell without affecting the ability of the cells to activate and induce the proliferation of NK cells.

The resulting mature, primed NK dendritic cells are then co-incubated with peripheral blood mononuclear cells, including NK cells. NK cells can be obtained from the NK dendritic cell cultures themselves or from various lymphoid tissues for use in the present invention. Such tissues include but are not limited to spleen, lymph nodes, and/or peripheral blood. The cells can be co-cultured with mature, primed dendritic cells as a mixed NK cell population or as a purified NK cells. NK cell purification can be achieved by positive, or negative selection, including but not limited to, the use of antibodies, either individually or in any combination, directed to CD16, CD56, CD2, CD3, HLA-DR, and the like.

By contacting NK cells with either mature or antigen primed NK dendritic cells a cell population comprising a 30- to 50-fold, or greater, increase in NK cells can be achieved. Typically, dendritic cells that have been cultured in the presence of GM-CSF alone or in combination with IL-4 can induce a 2- to 7-fold increase in the number of NK cells in a PBMC sample. Such methods can include contacting immature NK dendritic cells with BCG and IFNγ to prepare mature, primed dendritic cells. The immature dendritic cells can also be contacted with a predetermined antigen during or prior to maturation. The immature NK dendritic cells or mature NK dendritic cells can be enriched prior to maturation. In addition, NK cells can be enriched from the PBMCs prior to contacting with the NK dendritic cells.

In another aspect, methods are provided for combining the NK dendritic cells of the present invention with mature activated dendritic cells that present a predetermined antigen. Such a method can result in the induction of both a substantial proliferation of active NK cells, but also the induction of an antigen specific T cell response. The NK dendritic cells can be mixed with antigen presenting dendritic cells prior to admixing with, for example PBMCs, or the population of NK dendritic cells and antigen presenting dendritic cells can be added separately.

According to yet another aspect of the invention, NK dendritic cells can be preserved, e.g., by cryopreservation either before exposure to NK cells or prior to administration to an individual to be treated. Cryopreservation agents which can be used include but are not limited to dimethyl sulfoxide (DMSO), glycerol, polyvinylpyrrolidone, polyethylene glycol, albumin, dextran, sucrose, ethylene glycol, i-erythritol, D-ribitol, D-mannitol, D-sorbitol, i-inositol, D-lactose, choline chloride, amino acids, methanol, acetamide, glycerol monoacetate, and inorganic salts. Different cryoprotective agents and different cell types typically have different optimal cooling rates. The heat of fusion phase where water turns to ice typically should be minimal. The cooling procedure can be carried out by use of, e.g., a programmable freezing device or a methanol bath procedure. Programmable freezing apparatuses allow determination of optimal cooling rates and facilitate standard reproducible cooling. Programmable controlled-rate freezers such as Cryomed or Planar permit tuning of the freezing regimen to the desired cooling rate curve.

After thorough freezing, NK dendritic cells can be rapidly transferred to a long-term cryogenic storage vessel. In a typical embodiment, samples can be cryogenically stored in liquid nitrogen (−196° C.) or its vapor (−165° C.). Considerations and procedures for the manipulation, cryopreservation, and long term storage of hematopoietic stem cells, particularly from bone marrow or peripheral blood, is largely applicable to the NK dendritic cells of the present invention. A discussion of cryopreservation for hematopoietic stem cells can be found, for example, in the following references, incorporated by reference herein: Taylor et al., Cryobiology 27:269-78 (1990); Gorin, Clinics in Haematology 15:19-48 (1986); Bone-Marrow Conservation, Culture and Transplantation, Proceedings of a Panel, Moscow, Jul. 22-26, 1968, International Atomic Energy Agency, Vienna, pp. 107-186.

Frozen cells are typically thawed quickly (e.g., in a water bath maintained at about 37°-41° C.) and chilled immediately upon thawing. It may be desirable to treat the cells in order to prevent cellular clumping upon thawing. To prevent clumping, various procedures can be used, including but not limited to the addition before and/or after freezing of DNase (Spitzer et al., Cancer 45: 3075-85 (1980)), low molecular weight dextran and citrate, hydroxyethyl starch (Stiff et al., Cryobiology 20: 17-24 (1983)), and the like. The cryoprotective agent, if toxic in humans, should be removed prior to therapeutic use of the thawed NK dendritic cells. One way in which to remove the cryoprotective agent is by dilution to an insignificant concentration. Once frozen NK dendritic cells have been thawed and recovered, they can be used to activate T cells as described herein with respect to non-frozen NK dendritic cells.

In vivo Administration of Cell Populations

In another aspect of the invention, methods are provided for administration of mature, primed NK dendritic cells, or activated NK cells, or cell population containing such cells, to a subject in need of immunostimulation. Such cell populations can include both mature or antigen primed dendritic cell populations, in combination with NK dendritic cells, and/or activated NK cell populations. In certain embodiments, such methods are performed by obtaining low-adherence dendritic cell precursors or immature low-adherence dendritic cells cultured in the presence of GM-CSF and IL-15, differentiating and maturing those cells in the presence of BCG and IFNγ and optionally a predetermined antigen, to form a mature dendritic cell population capable of inducing the differentiation and proliferation of NK cells and an antigen specific T cell response. The immature dendritic cells can be contacted with antigen prior to or during maturation. Such mature, primed dendritic cells (NK dendritic cells) can be administered directly to a subject in need of immunostimulation.

In another embodiment, the NK dendritic cells, peripheral blood mononuclear cells (PBMCs), and the recipient subject have the same MHC (HLA) haplotype. Methods of determining the HLA haplotype of a subject are known in the art. In a related embodiment, the dendritic cells and/or PBMCs are allogeneic to the recipient subject. For example, the dendritic cells can be allogeneic to the PBMCs and the recipient, which have the same MHC (HLA) haplotype. The allogeneic cells are typically matched for at least one MHC allele (e.g., sharing at least one but not all MHC alleles). In a less typical embodiment, the dendritic cells, NK cells, and the recipient subject are all allogeneic with respect to each other, but all have at least one common MHC allele in common.

According to one embodiment, the peripheral blood mononuclear cells are obtained from the same subject from which the immature dendritic cells were obtained. After differentiation or maturation and activation of the NK cells in vitro, the autologous NK cells are administered to the subject to provoke and/or modulate an existing immune response. For example, NK cells can be administered, by intravenous infusion, at doses of about 10⁸-10⁹ cells/m² of body surface area (see, e.g., (1992)). Infusion can be repeated at desired intervals, for example, monthly. Recipients can be monitored during and after NK cell infusions for any evidence of adverse effects. In addition, the mature dendritic cells of the present invention can be administered with a combination of IL-2. The combination results in an increase in the number of NK cells induced by the dendritic cells of the present invention and an activation of the increased NK cell population by IL-2.

According to another embodiments, NK dendritic cell matured with BCG and IFNγ according to the present invention can be injected directly into a tumor, or other tissue containing a target antigen. Such mature cells can induce the activation and proliferation of natural killer cells in vivo around or within the tumor thereby increasing the killing of tumor cells susceptible to NK cell lysis.

The following examples are provided merely as illustrative of various aspects of the invention and shall not be construed to limit the invention in any way.

### EXAMPLE 1

In this example low adherence monocytic dendritic cell precursors were isolated by adherence to glass coated beads from previously frozen PBMCs, washed, and re-suspended in culture medium (X-VIVO-15®) supplemented with GM-CSF alone or in combination with IL-4 or IL-15. After culture for 5 days a portion of the immature low adherence monocytic dendritic cell precursors were matured with BCG and IFNγ, and the immature and mature low adherence monocytic dendritic cells were assayed for the presence of cell surface markers.

Briefly, cell populations enriched in low adherence monocytic dendritic cell precursors (approximately 5 to 10×10⁷ cells) were isolated by adherence to glass coat microcarrier beads in the presence of high concentrations of human serum albumin (at least 1%) and eluted with PBS and EDTA. The eluted cells were washed with 30 ml X-VIVO-15® and spun down at 1100 RPM for 8 min at 4° C. The washed cells were counted and re-suspended at 1.5×10⁶ cells/ml in X-VIVO-15®. Three aliquots of about 1.5×10⁷ cells were pelleted and re-suspended in X-VIVO-15® with 2% human serum albumin (HSA) supplemental with either:

(a.) GM-CSF alone (500 U/ml)

(b.) GM-CSF (500 U/ml)+IL-4 (500 U/ml)

(c.) GM-CSF (500 U/ml)+IL-15 (100 mg/ml)

The cells were transferred into T-25 flasks and cultured for about 5 days at 37° C., 5.5% CO₂ laying flat.

After about 5 days of culture non-adherent cells were transferred to a 50 ml conical centrifuge tube and washed twice with 10 ml PBS. If a substantial number of what visually appeared to be dendritic cells remained attached to the flask, 10 ml of PBS was added and the cells were placed back in the incubator for approximately an additional 15 min. The flask was then agitated to remove loosely adherent cells and the cells were pooled with the non-adherent cells collected previously. Pooled cells were centrifuged at 1100 RPM for 8 min at 4° C. and re-suspended in 10 ml X-VIVO-15®. The numbers of live and dead cells were determined by Trypan blue exclusion and 4 ml of the cells were split into two 15 ml conical centrifuge tubes for each treatment group. A portion of the cells were cultured for an additional 20 hrs in the presence of BCG (1:400 dil) and IFNγ (500 U/ml) in X-VIVO-15® plus 2% HSA to form mature DCs. The phenotype of the immature and mature dendritic cells was determined by analysis of the expression of cell surface markers (CD1a, CD86, and CD80) using a fluorescently labeled monoclonal antibodies specific for the markers. DCs were analyzed either on Day 5 as immature DCs or on Day 6 subsequent to 20 h of maturation with BCG (1:400 dil) and IFNγ (500 U/ml).

TABLE 1
Mean fluorescence intensity (mfi) and percentage
of positive cells (%) for various phenotypic markers,
for DCs cultured using different cytokines.

| | GM-CSF | | GM-CSF + IL-4 | | GM-CSF + IL-15 | |
|---|---|---|---|---|---|---|
| | immature | mature | immature | mature | immature | mature |
| CD1a | 128 | 51 | 16 | 11 | 314 | 113 |
| mfi | | | | | | |
| CD86 | 22 | 126 | 27 | 94 | 23 | 233 |
| mfi | | | | | | |
| CD80 | 24 | 46 | 17 | 21 | 24 | 58 |
| mfi | | | | | | |

Phenotypically the low-adherence dendritic cell precursors cultured in the presence of GM-CSF and IL-15 were determined to express the cell surface markers characteristic of dendritic cells. (Table 1). In particular, CD80, and CD86 expression were increased on the surface of mature DCs cultured in the presence of GM-CSF and IL-15 as compared to mature DCs grown in the presence of only GM-CSF or in the presence of GM-CSF and IL-4. No significant differences were observed in these markers between the immature DCs from all three culture conditions. Increased expression (Mean Fluorescence Intensity (MFI)) of CD1a on the surface of GM-CSF plus IL-15 generated immature and mature DCs were observed in comparison to DCs generated in GM-CSF alone or GM-CSF with IL-4.

### EXAMPLE 2

In this example low-adherence monocytic dendritic cell precursors were collected as described above and were cultured in the presence of GM-CSF alone, or in the presence of GM-CSF in combination with either IL-4 or IL-15. The cells were tested for viability and for the yield of immature dendritic cells. The yield of immature dendritic cells, on Day 5, as determined by live cell counts using Trypan blue exclusion from one donor (P052) is presented as an average from 3 experiments using low adherence monocytes that were either freshly isolated or cryopreserved prior to incubation in X-VIVO-15®, 2% HSA plus GM-CSF and IL-15. In a separate experiment the yield of DCs was also compared between immature DCs and mature DCs.

Briefly, the isolated low-adherence monocytic dendritic cell precursors were incubated at 1.5×10⁶ cells/ml in 10 ml X-VIVO-15® plus 2% human serum albumin supplemented with GM-CSF alone (500 U/ml), GM-CSF and IL-4 (500 U/ml), or GM-CSF and IL-15 (100 ng/ml) in a T25 flask. After 5 days the resulting cells were harvested and counted using Trypan blue exclusion. Immature low adherence DCs (2×10⁶ cells) were incubated in a single well of a 24-well plate in 2 ml media containing BCG (1:400 dil) and IFNγ (500 U/ml) for 20 h before harvesting and counting subsequent to Trypan blue staining.

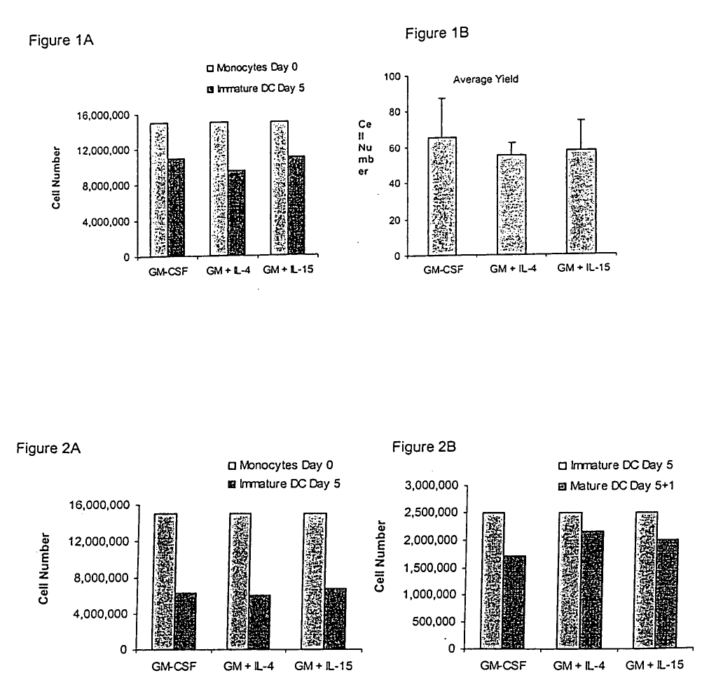

Culture and maturation of low-adherence monocytic dendritic cells in GM-CSF alone or in GM-CSF with either IL-4 or IL-15 resulted in approximately equivalent yields (approximately 63 to about 73% of input cells; FIGS. 1A and 1B). The viability of the cells produced was also approximately equal at about 90% viability. Low-adherence dendritic cell precursors from a second donor (P054) harvested after 5 days of culture in GM-CSF and IL-15 had comparable yields before (FIG. 2A) and after maturation (FIG. 2B) (approximately 40 to 45% of input cells for immature DCs and approximately 69% to 86% of input immature dendritic cells after maturation). Viability was approximately 90% as compared to GM-CSF alone or GM-CSF plus IL-4 DCs.

### EXAMPLE 3

In this example mature low-adhesion dendritic cells were tested for the release of cytokines. In particular, the amounts of GM-CSF, IL-12 (IL-12 subunit p70), TNF-α and IL-1β that were secreted by the cells was determined when they were cultured in the presence of GM-CSF alone and in the presence of GM-CSF in combination with either IL-4 or IL-15.

Briefly, low-adherence monocytic dendritic cell precursors were cultured as described above. The immature dendritic cells were loaded with KLH (40 μg/200 μl X-VIVO-15® or M1-A4 40 mer peptide (40 μg/200 μl X-VIVO-15®) then matured for 20 h with BCG (1:400 dil) and IFNγ (500 U/ml). Subsequently, supernatants were collected and run in duplicate on a BIOPLEX protein array system (BioRad) to quantify the amounts of GM-CSF, IL-12 p70, IL-1β, IL-10, and TNFα using the manufacturers protocols.

Both BCG and BCG plus IFNγ matured low-adherence dendritic cells cultured in GM-CSF plus IL-15 expressed higher levels of IL-12p70, TNFα, IL-1β and GM-CSF than DCs generated in either GM-CSF alone or GM-CSF supplemented with IL-4. Mature DCs cultured in GM-CSF supplemented with IL-15 expressed similar levels of IL-10 as mature DCs cultured in GM-CSF alone, but at higher levels than those cultured in GM-CSF supplemented with IL-4. (Table 2)

TABLE 2
Function of matured dendritic cells cultured in the
presence of various cytokines as measured by
cytokine release. The amount of cytokine is
expressed as pg cytokine/ml media.

| | GM-CSF | GM-CSF + | GM-CSF + |
|---|---|---|---|
| | Alone | IL-4 | IL-15 |
| GM-CSF | 74 | 47 | 181 |
| IL-12 p70 | 83 | 20 | 580 |
| IL-1β | 844 | 646 | 1,241 |
| IL-10 | 2,938 | 1,227 | 3,251 |
| TNFα | 23,146 | 8,090 | 36,000 |

### EXAMPLE 4

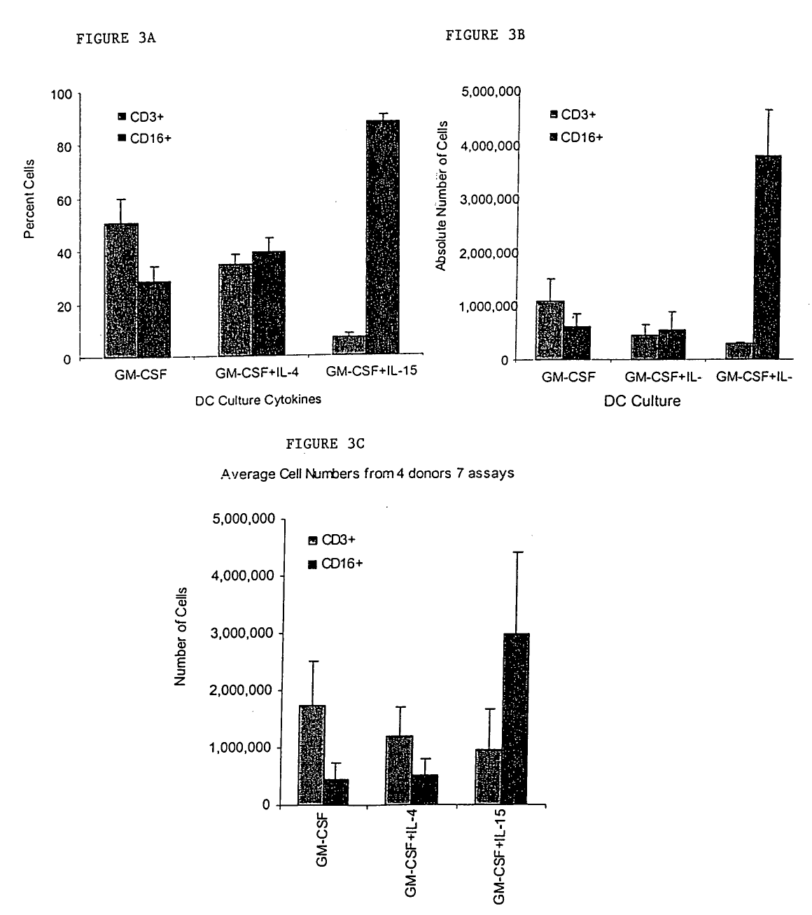

In this example matured low adhesion dendritic cells generated in GM-CSF and IL-15 were compared to DCs generated in GM-CSF alone or GM-CSF and IL-4 for their ability to stimulate T cell and/or NK cell expansion from peripheral blood mononuclear cells (PBMC) during an 8 or 9 day co-culture in T cell media (AIMV plus 5% human AB sera) supplemented on day 2 and subsequently every 2 to 3 days with 20 U/ml IL-2 and 5 ng/ml IL-15. The DCs were matured either with BCG (1:400 dil) alone or in combination with IFNγ (500 U/ml) after having been previously loaded with either 40 μg of either keyhole limpet hemocyanin (KLH) or a 40 mer peptide from the M1 protein of influenza A (M1-A4; (ProLeuThrLysGlyIleLeuGlyPheValPheThrLeuThrValProSerGluArgGlyLeuGlnArgArgArg PheValGlnAsnAlaLeuAsnGlyAsnGlyAspProAsnAsnMet; SEQ ID NO: 1)). Co-cultures were set up using each type of DC and autologous PBMC at a 1:10 DC:PBMC ratio. Eight days later, each cell line was analyzed for the percentage of CD3⁺/CD16⁻ (CD3⁺) and CD16⁺/CD3⁻ (CD16⁺) cells by flow cytometry and the absolute cell numbers of each population were calculated by multiplying the total cell culture counts by these percentages. The data from four separate co-cultures were averaged and are shown in FIG. 3A (percentage of cells expressing CD3 or CD16) and 3B (absolute numbers of CD3⁺ and CD16⁺ cell in the co-cultures by day 8). The combined average of seven similar experiments using four different doors is shown in FIG. 3C. These data demonstrate that DCs generated in GM-CSF plus IL-15 were consistently able to stimulate the expansion of NK cells to a much greater extent than DCs generated in GM-CSF alone or GM-CSF plus IL-4.

### EXAMPLE 5

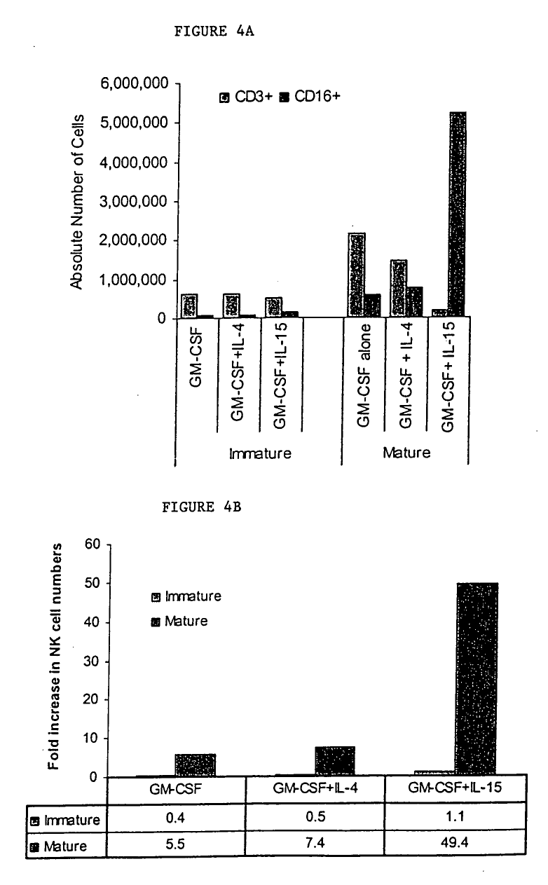

This example demonstrates that immature low-adherence DCs are not as effective at NK cell expansion as mature DCs. Briefly, low-adherence DC precursor cells were cultured in X-VIVO-15® plus 2% HSA supplemented with GM-CSF alone (500 U/ml), GM-CSF plus IL-4 (500 U/ml), or GM-CSF plus IL-15 (100 ng/ml), for 5 days before harvesting. One half of the DCs were matured with BCG (1:400 dil) plus IFNγ (500 U/ml) for a further 20 h. Immature and mature low-adherence DCs were then tested for their ability to expand NK cell by co-culturing with autologous PBMC at a 1:10 DC to PBMC ratio for 9 days. The resulting cell lines were harvested, counted and stained for CD3 (T cells) or CD16 (NK cells) expression using fluorochrome labeled monoclonal antibodies and analyzing by flow cytometry. The absolute numbers of each cell population was calculated by multiplying the percentage of CD3⁺ or CD16⁺ cell found in each cell line by the absolute number of cells in each co-culture. (FIG. 4) Only matured DCs generated in GM-CSF and IL-15 were able to expand NK cells to a significant degree (5.2×10⁶ CD16⁺ NK cells) as compared to mature DCs generated in GM-CSF alone (0.8×10⁶ CD16⁺ NK cells) or immature GM-CSF plus IL-15 generated DCs (0.1×10⁶ CD16⁺ NK cells). Mature NK dendritic cells generated in GM-CSF plus IL-15 were able to activate the initial population of NK cells contained in the PBMC and/or DC preparation from approximately 100,000 cells to 5 million cells; a 50-fold increase in 9 days (Table 3). DCs generated in GM-CSF and IL-4, or GM-CSF alone, or immature DCs generated under all three conditions had a maximum fold increase of only 7-fold over the same time frame.

TABLE 3
Immature low-adherence dendritic cells are not
as effective at NK cell expansion as mature
dendritic cells. Results are provided as the
fold-increase in NK cell numbers.

| | | GM-CSF + | GM-CSF + |
|---|---|---|---|
| | GM-CSF | IL-4 | IL-15 |
| Immature | 0.04 | 0.5 | 1.1 |
| Mature | 5.5 | 7.4 | 49.4 |

### EXAMPLE 6

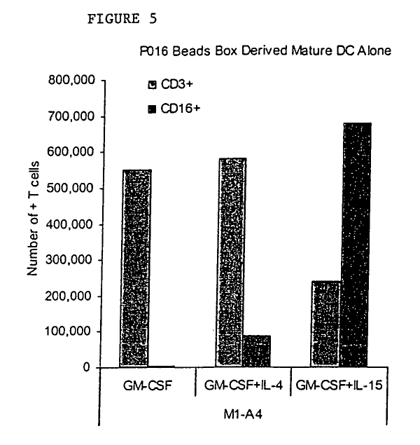

In this example it was demonstrated that NK cells already present in a low adherence NK dendritic cell precursor population expand rapidly after NK DC maturation and further culture in T cell medium. Briefly, mixed populations of cell containing low-adherence NK DC precursors and T cells were incubated for 5 days in X-VIVO-15® plus 2% HSA supplemented with GM-CSF alone (500 U/ml), GM-CSF plus IL-4 (500 U/ml), or GM-CSF plus IL-15 (100 ng/ml), before harvesting. The DCs were then loaded with Influenza A M1-4A 40mer peptide (SEQ ID NO: 1; 40 μg/200 μl X-VIVO-15®) or KLH (40 μg/200 μg/ml X-VIVO-15®) for 1 h prior to washing and maturation with BCG (1:400 dil) plus IFNγ (500 U/ml) for an 20 additional hours. The resulting DCs were stained for CD56 expression to determine how many NK cells were present in the preparation. The DCs cultured in GM-CSF alone contained 0.6%, GM-CSF plus IL-4 DCs contained 1.0%, and GM-CSF plus IL-15 contained 2% CD56⁺ NK cells. The matured low-adherence DCs were then washed, transferred to T cell media (AIM-V® plus 5% human AB sera) and 2.5×10⁶ cells were cultured for eight days in the presence of IL-2 (20 U/ml) and IL-15 (5 ng/ml). The resulting cell lines were harvested, counted and stained for CD3 (T cells) or CD 16 (NK cells) using fluorochrome labeled monoclonal antibodies and then analyzed by flow cytometry. The absolute numbers of each cell population was calculated by multiplying the percentage of CD3⁺ or CD 16⁺ cells found in each cell line by the absolute number of cells in each co-culture (FIG. 5). The starting numbers of CD56⁺ NK cells were 1500 for GM-CSF alone DC, 2500 for GM-CSF plus IL-4 DC and 5000 for GM-CSF plus IL-15 DCs. The expansion of NK cells after eight days culture in T cell media was 2.8-, 35.6-, and 136.0-fold respectively for these cell lines.

### EXAMPLE 7

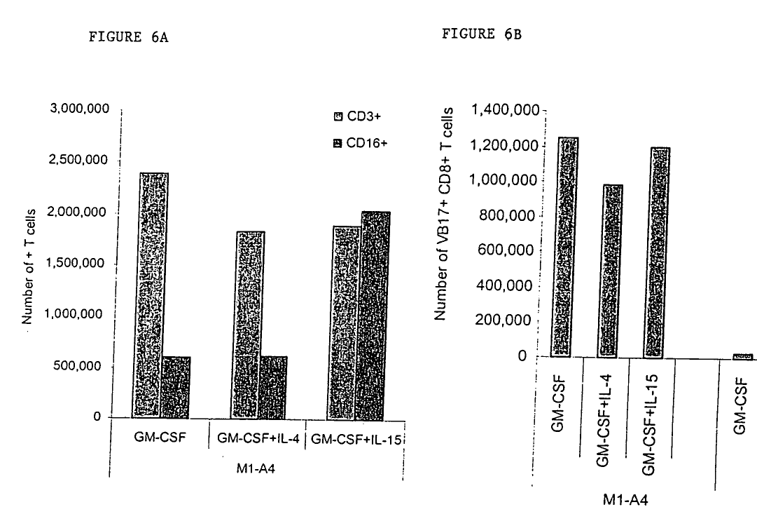

In this example low-adherence DCs cultured in various cytokines were tested for their ability to induce antigen specific T cell expansion and NK cell expansion. Low-adhesion monocytic dendritic cell precursors were isolated from an HLA-A2.1 donor and were incubated in X-VIVO-15® supplemented with 2% human serum albumin supplemented with either GM-CSF alone (500 U/ml), GM-CSF supplemented with IL-4 (500 U/ml), or GM-CSF with IL-15 (100 ng/ml), as described previously. After 5 days of culture the DCs were loaded with Influenza A M1-A4 40 mer peptide (SEQ ID NO.: 1; 40 μg/200 μl X-VIVO-15®) or KLH (40 μg/200 μl X-VIVO-15® for 1 h before washing and maturing with BCG (1:400 dil) and IFNγ (500 U/ml) for about 20 additional hours. Matured low-adherence dendritic cells were then co-cultured with autologous PBMC at a 1:10 DC to PBMC ratio in T cell media (AIM-V® plus 5% human AB sera) for eight days. The resulting cell lines were harvested, counted and stained for CD3 (T cells) or CD16 (NK cells) and Vβ17 and CD8 (M-1 protein specific CD8 T cells) using fluorochrome labeled monoclonal antibodies and then analyzed by flow cytometry. The absolute numbers of each cell population was calculated by multiplying the percentage of CD3⁺ and CD16⁺ cells (FIG. 6A), or Vβ17⁺/CD8⁺ cells (FIG. 6B) found in each cell line by the absolute number of cells in each co-culture. Low-adherence DCs generated in GM-CSF supplemented with IL-15 induced increased numbers of CD16⁺/CD3⁻ NK cells compared to DCs generated in GM-CSF alone or GM-CSF plus IL-4. However all three types of DCs were able to stimulate M1-specific expansion from the autologous PBMC population when first loaded with M1-A440 mer peptide in comparison to the control protein KLH.

PBMC were also stimulated with a mixture of low-adherence dendritic cells that had been cultured in GM-CSF and IL-15 and low-adherence dendritic cells that had been cultured in GM-CSF and IL-4. The stimulated T cell lines were tested, eight days later, for the percentage of cells expressing CD3 or CD56.

Briefly, low adherence monocytic dendritic cell precursors were cultured in media supplemented with either GM-CSF and IL-15 or GM-CSF and IL-4 as described above. GM-CSF+IL-15 generated dendritic cells (3.3×10⁴ cells) were mixed with GM-CSF plus IL-4 cultured DCs (6.6×10⁴ cells, loaded with M1-A4 protein, matured with BCG (1:400 dil) and IFNγ (500 U/ml) for 20 h before co-culturing with PBMCs for 8 days in AIM-V® plus 5% human AB sera supplemented with IL-2 (20 U/ml) and IL-15 (5 ng/ml) as described above. The resulting cells were stained with labeled antibody specific for CD3 and CD56 and the percentage of cells expressing CD3 or CD56 was determined by flow cytometry.

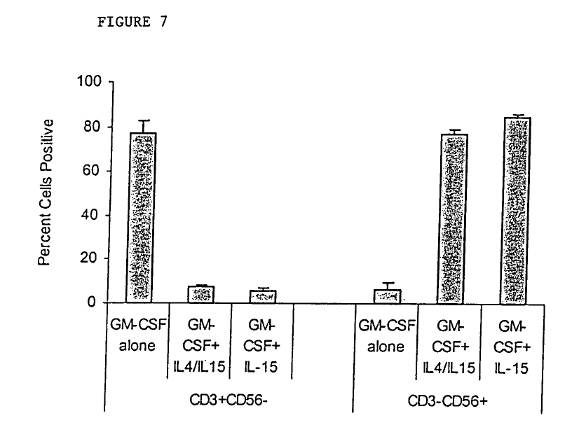

Mixing dendritic cells generated either in GM-CSF and IL-4 with dendritic cells cultured in GM-CSF and IL-15 induced or activated PBMCs into cells with characteristics similar to those generated with dendritic cells matured with GM-CSF and IL-15. (FIG. 7).

In addition, the number of PBMCs expressing a NK cell phenotype (CD16⁺CD56^{dim}) after co-culture with low-adherence mature, antigen loaded dendritic cells cultured in GM-CSF alone, or GM-CSF in combination with either IL-4 or IL-15 was also determined. Typically NK cells make up approximately 10 to 15% of the PBMCs from normal donors. PBMCs (1×10⁶ cells) were co-cultured with mature, antigen loaded low-adherence dendritic cells cultured in GM-CSF and IL-15. After 9 days in co-culture NK cells had expanded from approximately 100,000-150,000 cells to an average of approximately 4.3×10⁶ NK cells (CD16⁺CD56^{dim}) in two separate cultures. This represents a 30 to 40 fold increase in NK cell numbers over a 9 day period. In a similar experiment using low-adherence DCs generated in either GM-CSF alone or in combination with IL-4, the number of cells expressing NK markers were found to increase by only approximately 7 to 10 fold.

### EXAMPLE 8

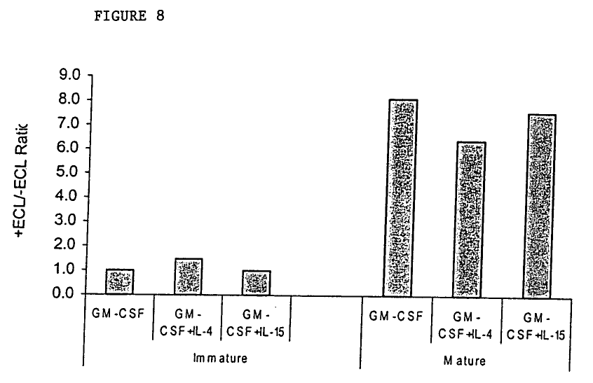

In the present example the ability of mature low-adherence dendritic cells cultured in the presence of GM-CSF and IL-15 to migrate in response to the chemokine MIP-3β (macrophage-inflammatory protein-3β) was compared with mature low-adherence dendritic cells cultured in the presence of either GM-CSF alone or in the presence of GM-CSF supplemented with IL-4. The chemokine MIP-3β binds the chemokine receptor CCR7 which is known to be upregulated on mature DCs. (FIG. 8).

Briefly, low-adhesion monocytic dendritic cell precursors were cultured in GM-CSF alone, or with GM-CSF in combination with either IL-4 or IL-15 as described above. The cells were tested for migration towards the chemokine MIP-3β (500 ng/ml) after maturation with BCG and IFNγ. The methods for maturation are described above. Low-adherence mature DCs (5×10⁴ cells) were placed in 6.5 mm transwells with 5 m pores inside the wells of a 24-well plate. Culture media (600 μl of X-VIVO-15® with or without 1.0 μg/ml (MIP-3β) was placed in the bottom chamber. The plates were incubated for 2 h at 37° C. in 5.5% CO₂. The cells that migrated through the filter were harvested from the bottom chamber, centrifuged and resuspended in 0.1 ml PBS with 1% paraformaldehyde. The cells were then counted on the flow cytometer for 30 sec using the high flow rate (60 μl/ml). The ratio of DCs migrating toward the chemokine MIP-3β versus background migration (no MIP-3β) was calculated and the result is depicted in FIG. 8. These data demonstrate that immature low-adherence DCs do not migrate in response to MIP-3β as expected for typical immature dendritic cells. However, the low-adherence mature DCs cultured in all three combinations of cytokines demonstrated similar chemotactic mobility towards the chemokine MIP-3β.

### EXAMPLE 9

In the present example the functional ability of the NK phenotype cells detected in PBMC that had been co-cultured with mature, antigen loaded, low-adherence DCs generated in the presence of GM-CSF and IL-15 was compared to similar PBMC that had been co-cultured with low-adherence DCs generated in either GM-CSF alone or GM-CSF in combination with IL-4. In particular, the ability of the NK cells to lyse the NK sensitive tumor cell line K562 was determined.

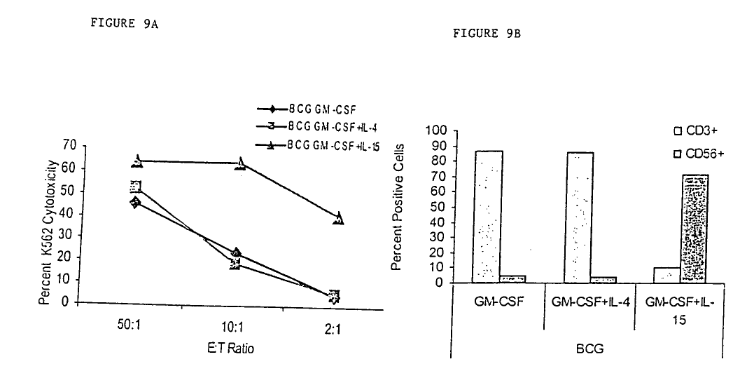

Briefly, PBMC were stimulated by co-culture with low-adherence dendritic cells cultured in either GM-CSF or in GM-CSF in combination with either IL-4 or IL-15 as described above and matured with BCG (1:400) to form effector cell lines. The cell lines were maintained in culture for one month in AIM-V® with 5% human AB sera supplemented with IL-2 (20 U/ml) and IL-15 (5 ng/ml). Lysis of tumor cells was determined by a standard 4 h ⁵¹Cr release assay by combining 5×10³ chromium labeled K562 tumor cells as targets per well with effector cells at a 50:1, 10:1 and 2:1 ratio. (FIG. 9A) At the lowest E:T ratio (2:1) GM-CSF plus IL-15 cell lines had 41% lysis against K562 targets while the lines generated in GM-CSF alone or GM-CSF plus IL-4 had only 5% K562 cell killing. Each cell line tested in the assay was also analyzed for the percentage of CD56⁺ NK cells versus CD3⁺ T cells by flow cytometry. The cells (approximately 1×10⁵) were stained with 2 μl anti-CD3-FITC, anti-CD56-CYC, and anti-CD16-PE label antibodies in a total volume of 100 μl FACS buffer for 15 min on ice. The cells were washed with 1 ml FACS buffer and resuspended in 0.250 ml PBS containing 1% paraformaldehyde before running on the FACsan flow cytometer. The data depicted in FIG. 9B is provided as the percentage of cells expressing either CD3 or CD56. Effector cell lines initiated with low-adherence DCs generated in GM-CSF plus IL-15 contained approximately 71% CD56^{dim} NK cells as compared to only 5% and 4% in effector cell lines initiated in low-adherence DCs generated in either GM-CSF alone or GM-CSF in combination with IL-4, respectively (FIG. 9B).

Overall these data unexpectedly demonstrated that monocytic dendritic cell precursors isolated by adhesion to glass coated beads in the presence of high concentrations of protein to block non-specific binding, cultured in the presence of GM-CSF and IL-15, and matured induce the generation of large numbers of natural killer cells. Dendritic cell precursors isolated by a typical method or by the same method, but cultured in the presence of GM-CSF alone or in the presence of GM-CSF and IL-4 induced PBMCs to produce substantial numbers of CD4⁺ T cells and CD8⁺ T cells, but few NK cells. Prior work has demonstrated that dendritic cells cultured in the presence of GM-CSF and IL-4 could activate resting human NK cells stimulating a two to four-fold increase in the number of NK cells (Ferlazzo et al., J. Exp. Med. 195:343-351). Using the methods of the present invention, at least 50%, or more, of the total number of cells stimulated by the dendritic cells matured in the presence of GM-CSF and IL-15 differentiate into NK cells. This percentage is even higher (70%) when only the NK cells already present in the NK-DC preparation are analyzed.

The previous examples are provided to illustrate but not to limit the scope of the claimed inventions. Other variants of the inventions will be readily apparent to those of ordinary skill in the art and encompassed by the appended claims. All publications, patents, patent applications and other references cited herein are hereby incorporated by reference.

What is claimed is:

1. A dendritic cell that can induce the activation and proliferation of natural killer cells.

2. The dendritic cell according to claim 1, wherein the dendritic cell is characterized by the expression of increased levels of CD80, and CD86 as compared to a mature dendritic cell cultured in the presence of granulocyte-macrophage colony stimulating factor (GM-CSF) and interleukin 4 (IL-4).

3. The dendritic cell according to claim 1, wherein the dendritic cell is characterized by the expression of an increased level of CD1a on the surface of the cell as compared to a dendritic cell cultured in the presence of GM-CSF and IL-4.

4. The dendritic cell according to claim 1, wherein the number of NK cells is increased by at least 10 fold from the initial NK cell numbers after at least seven days co-culture.

5. The dendritic cell according to claim 1, wherein the number of NK cell is increased by at least 30 fold.

6. The dendritic cell according to claim 1, wherein the dendritic cell is characterized by the expression of interleukin 12 (IL-12), tumor necrosis factor α (TNFα), and GM-CSF.

7. A method for the induction of the formation of a dendritic cell that can induce the activation and proliferation of natural killer cells, comprising:
providing a cell population comprising low-adherence monocytic dendritic precursor cells;
contacting the low-adherence monocytic dendritic precursor cells with an effective amount of granulocyte-monocyte colony stimulating factor (GM-CSF) and interleukin 15 (IL-15) to form low-adherence immature dendritic cells;
contacting the low-adherence immature dendritic cells with an effective amount of a dendritic cell maturation agent under culture conditions suitable for maturation of the low-adherence immature dendritic cells to form a low-adherence mature dendritic cell population.

8. The method according to claim 7, wherein the dendritic cell maturation agent is Bacillus Calmette-Guerin (BCG), lipopolysaccharide (LPS), TNFα, Interferon gamma (IFNγ), or combinations thereof.

9. The method according to claim 8, wherein the maturation agent is a combination of BCG and IFNγ.

10. The method according to claim 7, wherein the dendritic cells have been substantially purified.

11. The method according to claim 7, wherein the dendritic cells are characterized by the expression of increased levels of CD80, and CD86 as compared to a mature dendritic cell cultured in the presence of granulocyte-macrophage colony stimulating factor (GM-CSF) and interleukin 4 (IL-4).

12. The method according to claim 7, wherein the dendritic cells are characterized by the expression of an increased level of CD1a on the surface of the cell as compared to a dendritic cell cultured in the presence of GM-CSF and IL-4.

13. The method according to claim 7, wherein the dendritic cells are characterized by the expression of interleukin 12 (IL-12), tumor necrosis factor α (TNFα), and GM-CSF.

14. The method according to claim 7, wherein the dendritic cells are subsequently cryopreserved.

15. A method for inducing the activation and proliferation of natural killer (NK) cells, comprising:
contacting the NK cells with a dendritic cell that can induce the activation and proliferation of NK cells.

16. The method according to claim 15, wherein the dendritic cell is characterized by the expression of increased levels of CD80, and CD86 as compared to a mature dendritic cell cultured in the presence of granulocyte-macrophage colony stimulating factor (GM-CSF) and interleukin 4 (IL-4).

17. The method according to claim 15, wherein the dendritic cell is characterized by the expression of an increased level of CD1a on the surface of the cell as compared to a dendritic cell cultured in the presence of GM-CSF and IL-4.

18. The method according to claim 15, wherein the dendritic cells are characterized by the expression of interleukin 12 (IL-12), tumor necrosis factor α (TNFα), and GM-CSF.

19. The method according to claim 15, wherein the NK cells and the dendritic cell are contacted in vivo, ex vivo, or in vitro.

20. The method according to claim 15, wherein the NK cells are substantially isolated.

21. The method according to claim 15, wherein the NK cells are provided as a population of leukocytes.

22. The method according to claim 21, wherein the population of leukocytes are further contacted with antigen presenting dendritic cells.
